(19) United States
(12) Patent Application Publication
(10) Pub. No.: US 2006/0137699 A1
(43) Pub. Date: Jun. 29, 2006

(54) Providing data destination information to a medical device

(76) Inventors: Mark P. Moore, Redmond, WA (US); Kevin C. Drew, Snohomish, WA (US)

Correspondence Address: SHUMAKER & SIEFFERT, P. A., 8425 SEASONS PARKWAY, SUITE 105, ST. PAUL, MN, 55125, US

(21) Appl. No.: 11/022,342

(22) Filed: Dec. 23, 2004

(51) Int. Cl. A61B 5/04

(52) U.S. Cl. 128/920

(57) ABSTRACT

The disclosure describes a system in which medical devices, such as external defibrillators, vital signs monitors, or computers, collect medical event information during the treatment of a patient, and transmit the medical event information to a data destination, e.g., a computer at hospital or other medical facility. The system includes a remote server that stores data destination information for one or more data destinations, and transmits the data destinations information to the medical devices via a network. A medical device transmits medical event information to a data destination according to the data destination information that was received from the server for that destination. The data destination information for a particular destination may include, for example, information indicating a communication medium, a communication protocol, a telephone number, a network address, a network name, or login information used by a medical device to communicate with the destination.

### TECHNICAL FIELD

The invention relates to medical devices and, more particularly, to medical device communication.

### BACKGROUND

An external defibrillator delivers energy to a heart of a patient via electrodes placed upon the patient's chest. Often, external defibrillators are used to deliver energy in the form of a defibrillation pulse to a heart that is undergoing ventricular fibrillation and has lost its ability to contract. Ventricular fibrillation is particularly life threatening because activity within the ventricles of the heart is so uncoordinated that virtually no pumping of blood takes place. If untreated, the patient whose heart is undergoing fibrillation may die within a matter of minutes.

An electrical pulse delivered to a fibrillating heart may depolarize the heart and cause it to reestablish a normal sinus rhythm. In some cases, the patient may need multiple pulses, and the external defibrillator may deliver different quantities of energy with each defibrillation pulse. Further, the defibrillator may provide additional or alternative therapies to the patient, such as cardioversion or pacing therapy.

The defibrillator may also monitor the patient via the electrodes, and generate a record of the condition and treatment of the patient. For example, the defibrillator may record an electrocardiogram (ECG) of the patient sensed via the electrodes. The defibrillator may keep track of the therapy provided to the patient by, for example, recording the types and energy levels of defibrillation pulses delivered to the patient and the time at which these pulses were delivered. The defibrillator may also include a microphone to make an audio recording of the treatment of the patient. These and other types of information surrounding the treatment of the patient, i.e., medical event information, may be stored within a memory of the defibrillator.

In some cases, after the patient has been treated, the defibrillator may transmit the medical event information to another device. For example, after treating the patient with the defibrillator, paramedics or the like may transport the patient to a hospital or other medical facility for further treatment or monitoring. At the medical facility, the defibrillator may transmit the medical event information, or one or more reports generated from the medical event information, to a server or other type of computer located at the medical facility. A physician or the like may review the transmitted medical event information or reports prior to providing further treatment to the patient, and the transmitted medical event information or reports may become part of the patient's medical records.

Further, when the defibrillator is returned to a garage or other storage location where it resides between uses, it may transmit the medical event information, or one or more “run” reports generated from the medical event information, to a server or other type of computer located at the storage facility. Run reports may be used to evaluate the timeliness of the response to the medical emergency involving the patient, and the efficacy of the treatment provided to the patient. The computers at the medical facility and the storage facility are examples of destinations for the medical event information, i.e., data destinations.

Prior to transmission of the medical event information or reports to a destination, the defibrillator may need to be configured for communication with the destination and in some cases a network including the data destination. In many cases, a user of the defibrillator must determine how it should be configured and manually configure the defibrillator, which may be a time consuming process. Further, over time a single defibrillator may be used to treat many different patients that are transported to many different medical facilities, each of which may require the defibrillator to be configured differently for transmission of medical event information.

### SUMMARY

In general, the invention is directed to techniques for configuring a medical device that collects medical event information during treatment of a patient, such as an external defibrillators, vital signs monitor, or computer, to transmit the medical event information to a data destination. As discussed above, data destinations for collected medical event information may be computers at hospitals, other medical facilities, or a storage facility for the medical device. For example, in some embodiments, the medical device collects medical event information during treatment of patient in the field, e.g., during treatment by paramedics or first responders to a medical emergency involving the patient, and transmits the medical event information to a computer at a medical facility to which the patient is transported after treatment in the field, and a computer at a storage facility to which the medical device is returned after use. A system according to the invention includes a remote server that stores data destination information for one or more data destinations, and transmits the data destination information to one or more medical devices via a network, such as cellular telephone or other wireless network. The medical devices transmit medical event information to a data destination according to the data destination information that was received from the server for that destination.

The data destination information for a particular data destination may indicate a communication medium, a communication protocol, a telephone number, a network address, a network name, or login information to be used by a medical device to communicate with the destination. Additionally or alternatively, the data destination information may indicate a type of medical event information to be transmitted to the destination by a medical device, or a format in which the medical event information is to be transmitted to the destination. For example, the data destination information for a particular destination may indicate the type or format for a report including medical event information that is accepted by the facility that includes the data destination, and a medical device may generate a report, as directed by the data destination information, for transmission to the destination.

In some embodiments, the server stores and transmits data destination information for each of a plurality of data destinations. In such embodiments, a medical device may display a list of the data destinations, and receive a selection made from the list by a user. The list may be a list of names of the medical facilities and storage facilities that include the data destinations, which would be known to a user of the medical device. The user may, for example, select a data destination from a list by selecting the name of the medical facility to which the patient treated in the field is being transported. The medical device may then transmit medical event information according to the data destination information received from the server for the data destination, e.g., computer, at the selected medical facility.

In some embodiments, the server may receive modifications to the data destination information from a user. For example, the server may receive modifications to the data destination information via a client device used by the user and a network. The server may present a web interface including the data destination information currently stored by the server to the user via the client device, and the user may use the web interface to make modifications to the data destination information. The server may be, for example, administered by an emergency medical service (EMS), the paramedics and first responders of which use the medical devices that receive data destination information from the server. An administrative user from the EMS may modify the data destination information.

In some embodiments, the medical device stores the data destination information received from the server, and periodically connects to the server to receive updates to the stored data destination information from the server, e.g., modifications made by a user to the data destination information stored by the server as described above. As an example, the medical device may automatically connect to the server each time it is used, e.g., when it is powered on. When the medical device connects to the server, it may provide an indication of a version of the data destination information it currently stores, and receive any available updates to the data destination information since that version from the server based on the indication.

In one embodiment, the invention is directed to a medical device comprising a memory, a communication interface and a processor. The processor stores medical event information in the memory during treatment of a patient, receives data destination information from a remote server via a network and the communication interface, and transmits the medical event information to a data destination via the communication interface according to the data destination information.

In another embodiment, the invention is directed to a method comprising collecting medical event information during treatment of a patient with a medical device, receiving data destination information at the medical device from a remote server via a network, and transmitting the medical event information from the medical device to a data destination according to the data destination information.

In another embodiment, the invention is directed to a computer-readable medium comprising instructions. The instructions cause a programmable processor to collect medical event information during treatment of a patient with a medical device, receive data destination information at the medical device from a remote server via a network, and transmit medical event information from the medical device to a data destination according to the data destination information.

In another embodiment, the invention is directed to a system comprising a plurality of medical devices, and a server located remotely from the medical devices. The server stores data destination information for a data destination and transmits the data destination information to the medical devices via a network. Each of the medical devices collects medical event information during treatment of a respective patient, and transmits the medical event information to a data destination according to the data destination information.

The invention may provide advantages. For example, in embodiments in which a server provides data destination information to a medical device, a user of the medical device may not need to independently determine how the medical device should be configured for transmission of medical event information to a data destination. Further, a user may not have manually configure a medical device for transmission of medical event information to the data destination in embodiments in which the medical device partially or completely configures itself based on the data destination information received from the server. In some embodiments, a user need only select a data destination from a list displayed by the medical device, and the medical device will automatically configure itself for transmission of medical event information to the selected data destination.

As another example, by storing data destination information centrally within a server, the data destination information may be transmitted from the server to a plurality disparately located medical devices, such as a plurality of disparately located medical devices of an EMS system. Further, when the data destination information needs to be modified in such embodiments, e.g., due to a change in a network at a medical facility, a user need only modify the single instance of the data destination information stored by the server. Additionally, in some embodiments in which a medical device stores the data destination information received from the server, the server may need only transmit any modifications made to the data destination information stored by the server since the last time that the medical device connected to the server, reducing amount of data transmitted between the server and the medical device and the time that the medical device is connected to the server during each use of the medical device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an example system in which data destination information is provided to medical devices.

FIG. 2 is a block diagram illustrating an example medical device that receives data destination information.

FIG. 3 is conceptual diagram illustrating graphical user interface that may be provided to a user via a display of a medical device according to the invention.

FIG. 4 is a flow diagram illustrating an example method for updating data destination information stored by a server.

FIG. 5 is a flow diagram illustrating an example method for receiving data destination information from a remote server that may be performed by a medical device according to the invention.

FIG. 6 is a flow diagram illustrating an example method for transmitting medical event information to a data destination according to data destination information for the destination that may be performed by a medical device according to the invention.

### DETAILED DESCRIPTION

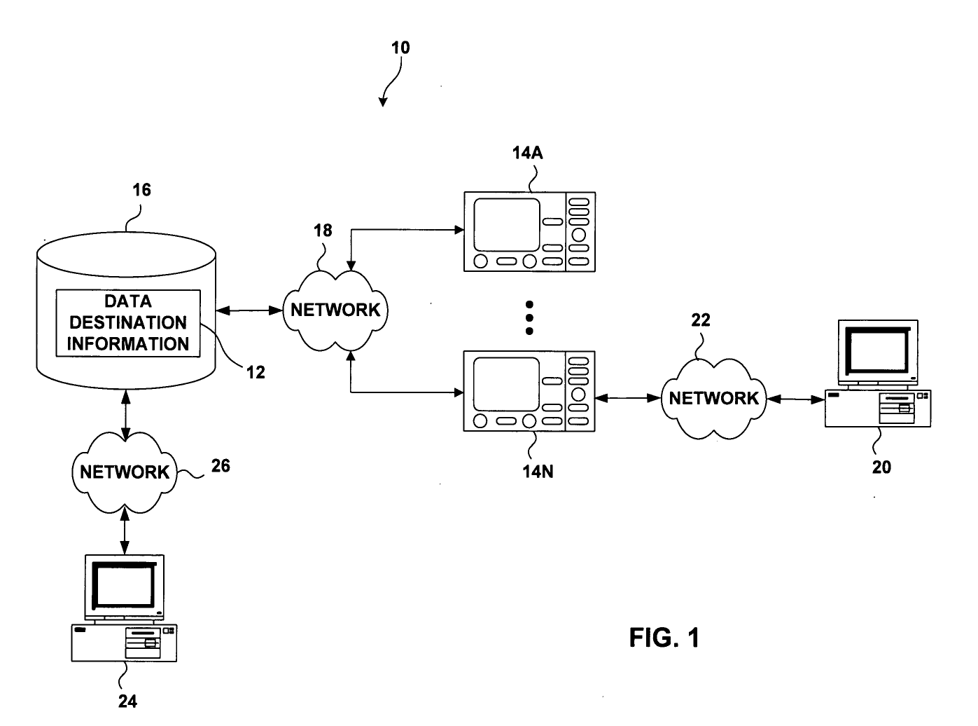

FIG. 1 is a block diagram illustrating an example system 10 in which data destination information 12 is provided to medical devices 14A-14N (collectively “medical devices 14”). More particularly, system 10 includes a server 16 that is located remotely from medical devices 14, stores data destination information 12, and transmits the data destination information to the medical devices via a network 18. Medical devices 14 may be disparately located. Network 18 may include one or more of a public switched telephone network (PSTN), a wireless network, a cellular telephone network, a wide area network (WAN), or the Internet. In exemplary embodiments, medical devices 14 receive the data destination information via wireless connections with server 16.

Medical devices 14 may be external defibrillators, vital signs monitors, computers, or any other type of implanted or external medical devices that collect medical event information during treatment of patients (not shown). Medical devices 14 may be used at different times and locations to treat different patients. Medical event information for a patient may include, for example, an electrocardiogram of the patient, a capnograph of the patient, a plethysmograph of the patient, a heart rate of the patient over time, a pulse rate of the patient over time, a blood oxygen saturation of the patient over time, a blood pressure of the patient over time, end tidal carbon dioxide measurements of the patient, measurements of the fraction of carbon dioxide in air inspired or expired by the patient, an indication of one or more therapies delivered to the patient, an indication of times at which the one or more therapies were delivered to the patient, or an audio recording. Medical event information collected by medical devices 14 may additionally or alternatively include demographic, medical history, or other information collected by users of the medical devices and entered into the medical devices via user interfaces thereof.

Medical devices 14 transmit medical event information collected for a patient to one or more data destinations, e.g., servers or other computers of hospitals, other medical facilities, or storage facilities for the medical devices. In some embodiments, medical devices 14 collect medical event information during the treatment of patients in the field, e.g., by paramedics or first responders to medical emergencies involving the patients, and transmit the medical event information to a computers at a medical facilities to which the patients are transported after treatment in the field, and computers at storage facilities to which the medical devices are returned after use. Although FIG. 1 illustrates a single computer 20, medical devices 14 may treat different patients at different times and locations, and may transmit medical event information to one or more of a plurality of computers associated with different medical and storage facilities. A plurality of medical devices 14 may collect medical event information during treatment of a single patient and individually transmit their collected medical event information to computer 20, or one of the medical devices, such as a computer, may collect medical event information from the other medical device for transmission to computer 20. When received by computer 20, medical event information for a patient may be reviewed by physicians or other caregivers at the medical facility that provide further treatment to the patient, and incorporated into a patient record for the patient.

As shown in FIG. 1, the medical devices may transmit the medical event information to the computer via a network 22, which may include a local area network (LAN) for a medical or storage facility, on which the computer resides. In some embodiments, medical devices 14 transmit the medical event information via a wireless networking connection with computer 20 and/or network 22. Further, the medical devices may transmit the medical event information to a data destination at a facility when they are located at the facility, or remotely from the facility. For example, medical devices 14 may transmit medical event information for a patient to a data destination of a medical facility when they are located at the medical facility, e.g. via a local wireless connection established when the patient arrives at the medical facility and is “handed off” to the caregivers at the medical facility. Alternatively, the medical devices may transmit medical event information for a patient to the data destination when they are located remotely from the medical facility, e.g. via a remote wireless connection established during treatment of the patient in the field or transportation of the patient to the medical facility.

Medical devices 14 transmit medical event information to computer 20 according to data destination information 12 received from server 16. Data destination information 12 may include information used to configure medical devices 14 for communication with computer 20, e.g., to configure respective communication interfaces of the medical devices for communication with computer 20 via network 22. For example, data destination information 12 may indicate a communication medium, a communication protocol, a telephone number, a network address, a network name, or login information to be used by medical devices 14 to communicate with computer 20.

Additionally or alternatively, data destination information 12 may indicate a type of medical event information to be transmitted to computer 20 by medical devices 14, or a format in which the medical event information is to be transmitted to the computer by the medical devices. For example, data destination information 12 may indicate the type or format for a report including medical event information that is accepted by the medical facility that includes computer 20, and medical devices 14 may generate a report, as directed by the data destination information, for transmission to computer 20. Because server 16 provides data destination information 12 to medical devices 14, users of the medical devices may not need to independently determine how the medical devices and the medical event information should be configured for transmission of the medical event information to computer 20.

In some embodiments, medical devices 14 display at least some of data destination information 12 received from server 16. Users of medical devices 14 may manually configure the medical devices for transmission of medical event information to computer 20 based on the displayed data destination information. In some embodiments, however, medical devices 14 at least partially configure themselves for transmission of medical event information to computer 20 based on the data destination information 12 received from server 16. In such embodiments, the medical devices may nonetheless display data destination information 12 that requires action by a user of the medical device, such as data destination information reminding a user to transmit medical event information to computer 20 and indicating a physical location within a medical or other facility at which a connection with computer 20 should be established, or a physical medium by which the connection to computer should be established. For example, a medical device 14 may display data destination information 12 directing a user to a location within a facility at which a wired or wireless connection with network 22 may be established for the purpose of transmitting medical event information to computer 20.

A user may modify, i.e., add to, change or delete, data destination information 12 stored by server 16. A user may need to modify data destination information 12 stored by server 16 for a variety of reasons. For example, new medical or storage facilities may be built in an area, leading to the creation of new destinations for medical event data. A user may add data destination information for the new destinations to information 12 stored by server 16. Further, a change in the networking infrastructure at an existing medical or storage facility may require a user to modify the data destination information 12 stored by server 16 for an existing data destination. By storing data destination information 12 within a central server 16 and transmitting it to a plurality of medical devices 14, the user may need only modify the single instance of data destination information stored by the server when data destinations change, rather than individually reconfiguring the plurality of medical devices, or individually informing the user of the plurality of medical devices of the change by other means.

In the example illustrated by FIG. 1, the user creates or modifies data destination information 12 using a client device 24 that is coupled to server 16 via a network 26. Client device 24 may be, for example, any type of handheld, laptop, or desktop computer. Network 26 may include one or more of a PSTN, a wireless network, a cellular telephone network, a LAN, a WAN, or the Internet. Server 16 may present a web interface including any data destination information 12 currently stored by the server to the user via client device 24, and the user may use the web interface to make modifications to the data destination information. Server 16 may present the web interface via a web browser run by client device 24.

By storing data destination information 12 centrally within server 16, the data destination information may be transmitted from the server to a plurality disparately located medical devices 14, such as a plurality of disparately located medical devices used by paramedics and first responders an emergency medical service (EMS) system. Server 16 may be administered by the EMS, e.g., an administrative user from the EMS system may use client device 24 to create modify the data destination information stored by server 12. In this manner, an EMS system may ensure that its medical devices 14 have access to current data destination information, and can be thereby be configured to transmit medical event information to any of a plurality of data destinations in a geographical area, without relying on users of the medical devices to independently determine how the medical devices should be configured.

In some embodiments, medical devices 14 connect to server 16, and download whatever current data destination information 12 is stored by server 16 for a data destination when the medical devices are used to transmit medical event information to that data destination. In such embodiments, medical devices 14 may connect to server 16 automatically, e.g., in response to an attempt to transmit medical event information or another event, or in response to an input from a user directing the medical device to connect to the server.

In other embodiments, medical devices 14 store data destination information 12 received from server 16. In such embodiments, medical devices 14 may periodically connect to the server to receive updates to the stored data destination information, e.g., modifications made by a user to the data destination information stored by the server as described above. Medical devices 14 may connect to server 16 automatically or in response to a user input. As an example, the medical devices may automatically connect to the server each time they are used, e.g., powered on. In other embodiments, server 16 periodically polls medical devices 14, and thereby controls when medical devices 14 connect to server 16.

When medical devices 14 connect to server 16, they may provide an indication of a version of data destination information 12 they currently store, e.g., an indication of the last update received or time of the last update. When server 16 receives a version indication from one of medical devices 14, the server may transmit any available updates to data destination information 12 since the indicated version to the medical device. In this manner, server 16 may need only transmit any modifications made to medical facility information 12 stored by the server since the last time that the medical device connected to the server, reducing amount of data transmitted between server 16 and medical device 14 and the amount of time that the medical devices are connected to the server during each use of the medical device.

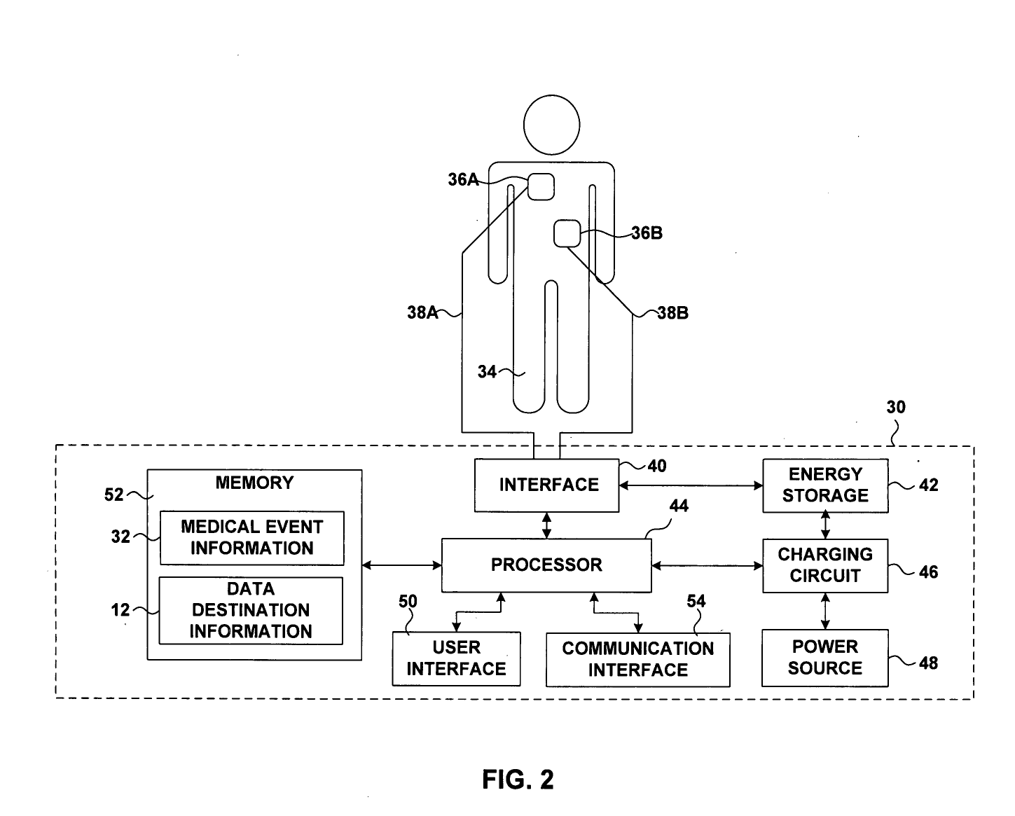

FIG. 2 is a block diagram illustrating an external defibrillator 30 that receives medical facility information 12 from server 16 (FIG. 1). Defibrillator 30 is an example of a medical device 14 (FIG. 1) that collects medical event information 32 during treatment of a patient 34, and transmits the medical event information to a data destination, e.g., computer 20 (FIG. 1) according to the data destination information.

In FIG. 2, defibrillator 30 is shown coupled to patient 34 by electrodes 36A and 36B (collectively “electrodes 36”). Although two electrodes 36 are shown in FIG. 2, defibrillator 30 may be coupled to patient 34 by any number of electrodes. In some embodiments, for example, defibrillator 30 is coupled to patient 34 by twelve or more electrodes 36. Defibrillator 30 is coupled to patient 34 in order to facilitate the treatment of patient 34, e.g., sensing electrical activity of the heart of patient 34 and delivering defibrillation pulses to patient 34 via electrodes 36. Defibrillator 30 need not be coupled to patient 34 when receiving data destination information 12 from server 16 (FIG. 1) or transmitting medical event information to computer 20 (FIG. 1).

Electrodes 36 may include hand-held electrode paddles or adhesive electrode pads placed on the skin of patient 34. Electrodes 36 are coupled to defibrillator 30 via respective conductors 38A and 38B (collectively “conductors 38”) and an interface 40. In a typical application, interface 40 includes a receptacle, and conductors 38 plug into the receptacle.

Interface 40 includes a switch (not shown in FIG. 2) that, when activated, couples an energy storage circuit 42 to electrodes 36. Energy storage circuit 42 stores the energy to be delivered to patient 34 in the form of a defibrillation pulse. The switch may be of conventional design and may be formed, for example, of electrically operated relays. Alternatively, the switch may comprise an arrangement of solid-state devices such as silicon-controlled rectifiers or insulated gate bipolar transistors.

Energy storage circuit 42 includes components, such as one or more capacitors, that store the energy to be delivered to patient 34 via electrodes 36. Before a defibrillation pulse may be delivered to patient 34, energy storage circuit 42 must be charged. A processor 44 directs a charging circuit 46 to charge energy storage circuit 42 to a high voltage level. Charging circuit 46 comprises, for example, a flyback charger that transfers energy from a power source 48 to energy storage circuit 42.

Defibrillator 30 may be a manual defibrillator or an AED. Where defibrillator 30 is a manual defibrillator, a user of defibrillator 30 may select an energy level for each defibrillation pulse delivered to patient 34. Processor 44 may receive the selection made by the user via a user interface 50, which may include input devices, such as a keypad and various buttons or dials, and output devices, such as various indicator lights, a cathode ray tube (CRT), light emitting diode (LED), or liquid crystal display (LCD) screen, and a speaker. Where defibrillator 30 is an AED, processor 44 may select an energy level from a preprogrammed progression of energy levels stored in a memory 52 based on the number of defibrillation pulses already delivered to patient 34.

When the energy stored in energy storage circuit 42 reaches the desired energy level, processor 44 controls user interface 50 to provide an indication to the user that defibrillator 30 is ready to deliver a defibrillation pulse to patient 34, such as an indicator light or a voice prompt. The defibrillation pulse may be delivered manually or automatically. Where the defibrillation pulse is delivered manually, the user may direct processor 24 to deliver the defibrillation pulse via user interface 40 by, for example pressing a button. In either case, processor 44 activates the switches of interface 40 to electrically connect energy storage circuit 42 to electrodes 36, and thereby deliver the defibrillation pulse to patient 34.

Processor 44 may modulate the defibrillation pulse delivered to patient 34. Processor 44 may, for example, control the switches of interface 40 to regulate the shape and width of the pulse. Processor 44 may control the switches to modulate the pulse to, for example, provide a multiphasic pulse, such as a biphasic truncated exponential pulse, as is known in the art.

Processor 44 may perform other functions as well, such as monitoring electrical activity of the heart of patient 34 sensed via electrodes 36. Processor 44 may determine whether the heart of patient 34 is fibrillating based upon the sensed electrical activity in order to determine whether a defibrillation pulse should be delivered to patient 34. Where a defibrillation pulse has already been delivered, processor 44 may evaluate the efficacy of the delivered defibrillation pulse by determining if the heart is still fibrillating in order to determine whether an additional defibrillation pulse is warranted. Processor 44 may automatically deliver defibrillation pulses based on these determinations, or may advise the caregiver of these determinations via user interface 50. Processor 44 may display an electrocardiogram (ECG) that reflects the sensed electrical activity via user interface 50.

Processor 44 may store an indication of the time of delivery of each defibrillation pulse delivered to patient 34 as medical event information 32 within memory 52 for patient 34. Processor 44 may also store the energy level of each pulse and other characteristics of each pulse, such as the width, amplitude, or shape, as medical event information 32 for patient 34. Processor 44 may also store a digital representation of the ECG, or a heart rate over time determined based on the electrical activity of the heart of patient 34 detected via electrodes 36 as medical event information 32 for patient 34. Further, processor 44 may control delivery of other types of therapy to patient 34 via electrodes 36, such as cardioversion or pacing therapy, and store information describing the times that such therapies were delivered and parameters of such therapies, such as cardioversion pulse energy levels and pacing rates, as medical event information 32 for patient 34.

User interface 50 may include a microphone (not shown) that detects sounds in the vicinity of defibrillator 30. Processor 44 may receive signals from the microphone and store an audio recording that includes these signals as medical event information 32 for patient 34. The audio recording may include verbal notations of a user of defibrillator 30, or conversations between the user and patient 34.

The user may mark the time of the occurrence of various events, such as the delivery of drugs or the administration of cardiopulmonary resuscitation (CPR), during the treatment of patient 34 by, for example, pressing a key or button of user interface 50 at the time when the event occurred. These event markers may also be included within medical event information 32 for patient 34. Where defibrillator 30 is more fully featured, e.g., a manual paramedic or hospital defibrillator, defibrillator 30 may also include additional sensors (not shown) coupled to processor 44, such as sensors to measure blood oxygen saturation, blood pressure, respiration, and the amount of oxygen or carbon dioxide in the air inhaled or exhaled by patient 34. Processor 44 may also store the signals generated by these sensors within memory 52 as medical event information 32 for patient 34. In other words, as examples, processor 44 may also store any of a capnograph, a plethysmograph, a blood oxygen saturation over time, a blood pressure over time, a pulse rate over time determined based on measured blood pressure, end tidal carbon dioxide measurements, and/or measurements of the fraction of carbon dioxide in air inspired or expired within memory 52 as medical event information 32 for patient 34. Processor 44 may begin to store medical event information 32 when defibrillator 30 is powered on to respond to a medical emergency.

Processor 44 receives data destination information 12 from server 16 (FIG. 1), and transmits medical event information 32 to one or more data destinations, such as computer 20 (FIG. 1), via a communication interface 54. In the illustrated example, processor 44 stores data destination information 12 in memory 52. Processor 44 may receive and store data destination information 12 for a plurality of data destinations. However, in some embodiments, processor 44 does not store the data destination information in memory 52, and instead downloads data destination information 12 for a particular data destination from server 16 as needed.

Communication interface 54 may include any one or more of a variety of circuits configured or configurable for communication via any of a variety of wired or wireless media, and according to any of a variety of protocols or standards. For example, communication interface 54 may include a cellular telephone transceiver capable of communicating according to one or more cellular telephone communication protocols, such as the North American Digital Cellular standards (IS-54, IS-88, IS-95, IS-136, or IS-637) developed by the Telecommunications Industry Association and Electronics Industry Alliance (TIA-EIA), the Global System for Mobile Communications (GSM) standards developed by the Conference of European Posts and Telegraphs (CEPT), the Personal Digital Cellular (PDC) standards provided by NTT DoCoMo, and/or the Mobile IP standards (RFC 2002) developed by the Internet Engineering Task Force (IETF).

In some embodiments, communication interface 54 may include an integrated circuit or circuit card with circuitry configured to be coupled to and communicate with or via a LAN or wireless LAN (WLAN), e.g., a wired or wireless network interface card. For example, communication interface 54 may be configured to communicate via a wired or wireless connection according to one or more of the 802 specification sets promulgated by the Institute of Electrical and Electronics Engineers (IEEE). In other embodiments, communication interface includes a modem for communication via a PSTN.

Additionally or alternatively, communication interface 54 may be configured to communicate via a wireless connection according to the Bluetooth specification set, which was promulgated by the Bluetooth Special Interest Group (SIG), and is available for download at http://www.bluetooth.org, or one or more of the specifications promulgated by the Infrared Data Association (IrDA). Further, communication interface 54 may include circuits to communicate data serially via a wired connection according to one of the RS-232, universal serial bus (USB), or IEEE 1394 standards.

In some embodiments, processor 44 configures communication interface 54. Processor 44 may configure communication interface 54 differently for communication with server 16 and each of a plurality of data destinations, e.g., computers 20. For example, processor 44 may activate a cellular telephone transceiver of communication interface 54 for communication with server 16, wireless network interface circuitry of the communication interface that is compliant with one or more of the IEEE 802.11 specification sets for communication with a network 22 and computer 20 at a hospital, and circuitry of the communication interface that is compliant with the Bluetooth specification set for communication with a computer 20 at the storage facility for defibrillator 30. Processor 44 may configure communication interface 54 for communication with a particular data destination based on data destination information 12 received from server 16 for that destination.

Processor 44 may, for example, include one or more of a microprocessor, digital signal processor (DSP), application specific integrated circuit (ASIC), field programmable gate array (FPGA), or other logic circuitry. In addition to data destination information 12 and medical event information 32, memory 52 may include program instructions that cause processor 44 to perform the functions attributed to processor 44 herein. Accordingly, the invention also contemplates computer-readable media storing instructions to cause processor 44 to provide the functionality described herein. Memory 52 may include any of a variety of solid state, magnetic or optical media, such as random access memory (RAM), read-only memory (ROM), CD-ROM, magnetic disk, electrically erasable programmable ROM (EEPROM), or flash memory.

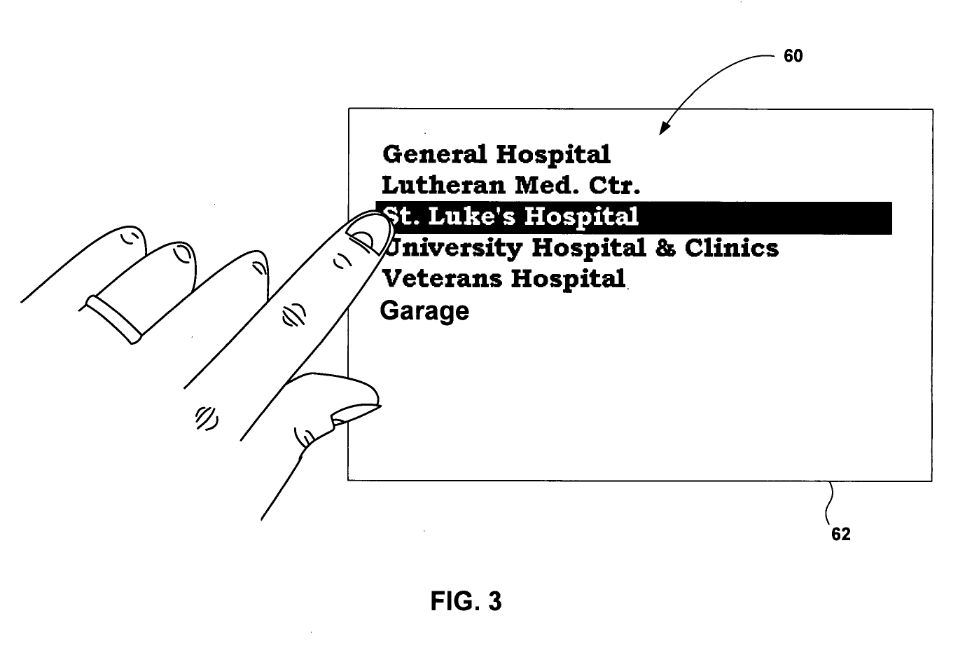

FIG. 3 is conceptual diagram illustrating a graphical user interface 60 that processor 44 may provide to a user of defibrillator 30 via a display 62 of the defibrillator. As described above, server 16 (FIG. 1) may store data destination information 12 (FIG. 1) for each of a plurality of data destinations. Graphical user interface 60 comprises a list of data destinations and, more particularly, a list of names of the medical and storage facilities that include the data destinations.

A user of defibrillator 30 may select a data destination to which defibrillator 30 is to transmit medical event information 32 from the list. In the illustrated embodiment, display 62 is a touch-screen display, and the user of defibrillator selects a data destination by touching display. In other embodiments, the user may use other components of user interface 50 (FIG. 2) to make a selection from the list, such as a keyboard, a mouse, or other pointing device.

The names in the list are names by which a user of defibrillator 30 would identify the facility. Consequently, if the user transports patient 34 to St. Luke's Hospital and wishes to transmit medical event information 32 for the patient to a computer 20 of the hospital, the user may simply select “St. Luke's Hospital” from the list. If the user later wishes to transmit the medical event information to a computer 20 at the storage facility of the defibrillator, e.g., for generation of a run report, the user may select “Garage” from the list.

In embodiments in which processor 44 stores data destination information 12 received from server 16 in memory 52, processor 44 may retrieve the data destination information 12 for the selected destination from memory 52 in response to receiving the selection. In other embodiments, processor 44 may control communication interface 54 to connect to server 16 in response to receiving the selection, and may download data destination information 12 for the selected destination from server 16.

In either case, processor 44 may display at least some of data destination information 12 for the selected destination via display 62. The user of defibrillator 30 may manually configure the defibrillator for transmission of medical event information to the selected data destination based on the displayed data destination information. For example, processor 44 may display data destination information 12 via display 62 that directs the user to a location within the selected facility at which a wired or wireless connection with a network 22 may be established for the purpose of transmitting medical event information to a computer 20. In some embodiments, however, processor 44 at least partially configures defibrillator 30 for transmission of medical event information to the selected data destination automatically based on the data destination information 12 for the selected destination.

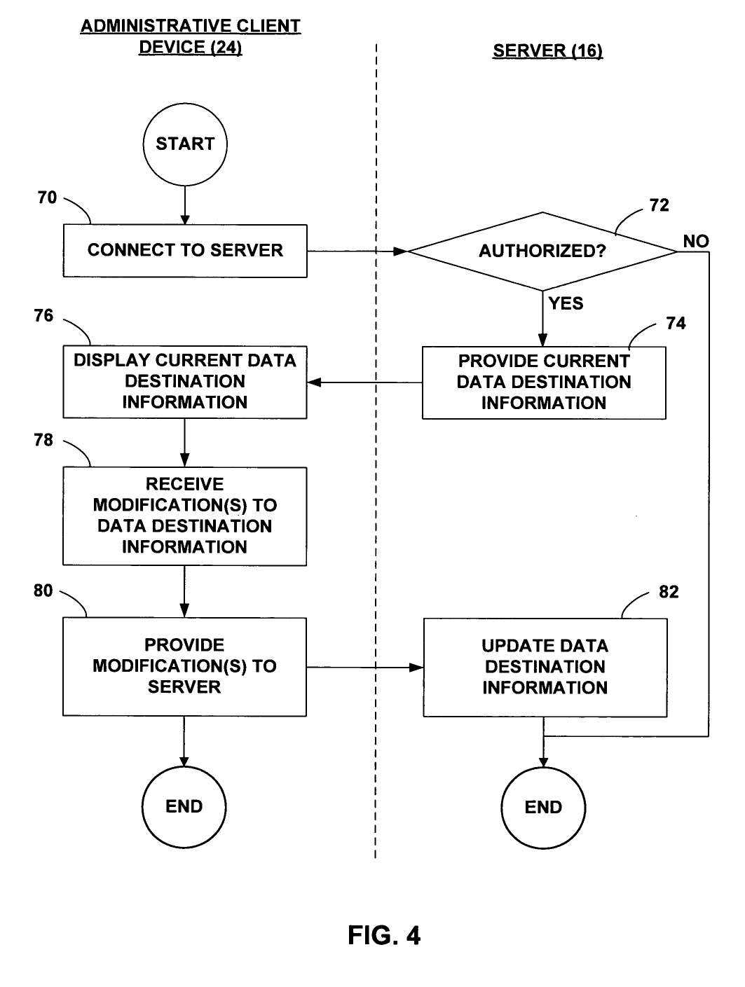

FIG. 4 is a flow diagram illustrating an example method for updating data destination information 12 stored by a server 16. According to the example method, an administrative user, such as an administrative user of an EMS system, directs client device 24 to connect to server 16 (70). Client device 24 may provide a web browser, and the user may enter a uniform resource locator (URL) associated with server 16 to cause the client device 24 to connect to server 16. Server 16 may request login information from the user, and determine if the user is authorized as an administrator for server 16, e.g., to create and modify data destination information 12, based on the login information (72). If the user is authorized, server 16 provides whatever data destination information is currently stored by the server to client device 24 (74).

Client device 76 displays the current data destination information 12 (76), receives modifications made to the data destination information by the user (78), and provides the modifications to server 16 (80) via the web browser. Server 16 updates the data destination information 12 stored thereon based on the received modifications (82). For example, server 16 may modify the stored data destination information in accordance with the modifications received from client device 24, or may store the received modifications for later distributions to medical devices 14, such as defibrillator 30.

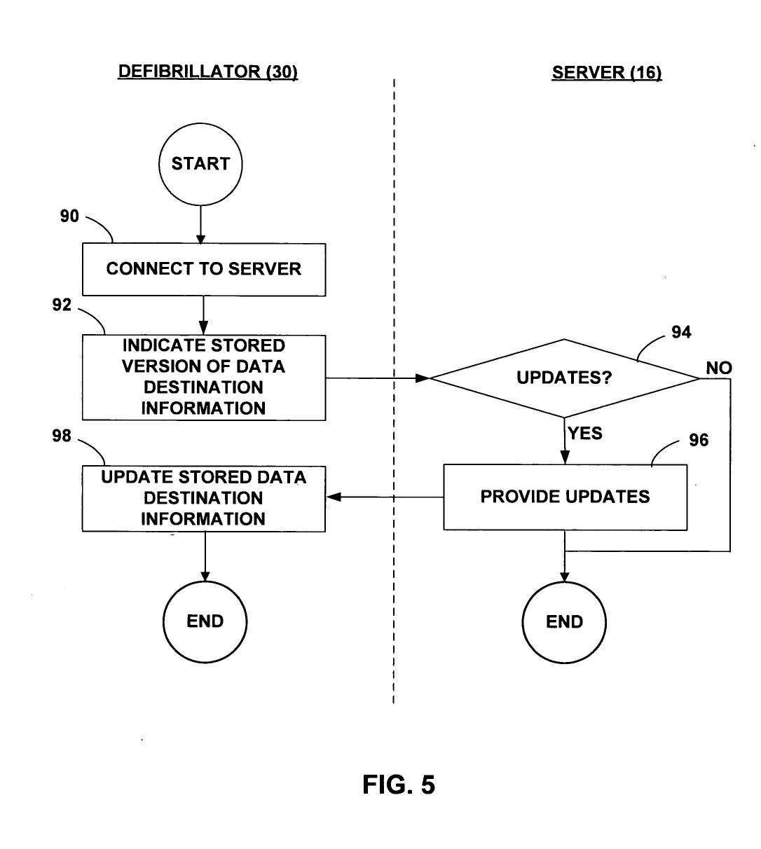

FIG. 5 is a flow diagram illustrating an example method for receiving data destination information 12 from a remote server 16 that may be performed by a medical device 14, such as defibrillator 30, according to the invention. More particularly, FIG. 5 illustrates a method that may be employed by medical devices 14 that store data destination information 12 received from server 16, and periodically receive updates to the data destination from the server. In other embodiments, as described above, medical devices 14 do not store data destination information 12, and instead download the data destination information currently stored by server 16 from the server as needed.

According to the example method, defibrillator 30 connects to server 16 via a network 18 (90). Defibrillator 30 may connect to server when directed by a user via a user interface 50, or automatically upon the occurrence of an event, such as being powered on. In other embodiments, server 16 may connect to defibrillator 30, e.g., poll the defibrillator. In either case, the connection between defibrillator 30 and server 16 may be established periodically so that defibrillator 30 receives up to date data destination information 12 from the server.

When connected to server 16, defibrillator 30 provides an indication of the version of data destination information that it currently stored in a memory 52 to the server (92). Defibrillator 30 may, for example, provide an indication of the date and time at which the defibrillator last received an update to data destination information 12 to server 16. In other embodiments, server 16 may store information indicating the version of data destination information that it currently stored by defibrillator 30, and the defibrillator consequently need not provide such an indication to the server.

Based on the stored or received version indication, server 16 determines whether there are any updates to data destination information 12 available for defibrillator 30 (94). If updates are available, server 16 transmits them to defibrillator 30 (96). Server 16 may, for example, transmit the entire current version of data destination information 12 stored by the server, or may only transmit modifications made by a user to data destination information 12 since the last update of defibrillator 30. In either case, defibrillator 30 updates data destination information 12 stored in memory based on the update received from server 16 (98).

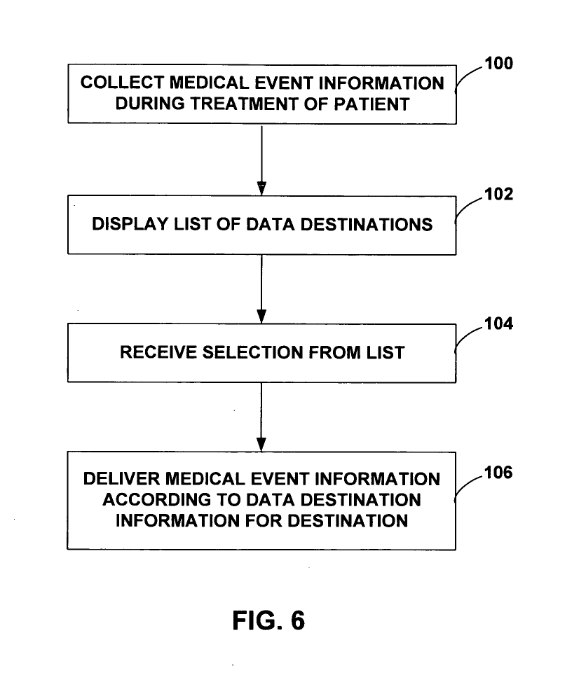

FIG. 6 is a flow diagram illustrating an example method for transmitting medical event information 32 to a data destination according to data destination information for that destination that may be performed by a medical device 14, such as defibrillator 30, according to the invention. According to the example method, defibrillator 30 collects medical event information 32 during treatment of patient 34 (100). Defibrillator 30 also displays a list of data destinations via a display 62 as described above with reference to FIG. 3 (102). When a user of defibrillator 30 wishes to transmit the medical event information to a data destination, the user selects the data destination from the list, and defibrillator 30 receives the selection (104). For example, when the user wishes to transmit the medical event information to a medical facility to which the patient has been transported, the user may select the name of the medical facility from the list.

In some embodiments, defibrillator 30 displays at least some of the data destination via display 62, and the user of the defibrillator at least partially configures the defibrillator based on the displayed information. In some embodiments, defibrillator 30 at least partially configures itself for communication with the data destination based on the data destination information. In either case, defibrillator 30 delivers medical event information 32 to the selected data destination according to data destination information 12 received from server 16 for that destination (106).

Various embodiments of the invention have been described. However, one skilled in the art will appreciate that various modifications may be made to the described embodiments without departing from the scope of the invention. For example, although described herein primarily in the context of external defibrillators, medical devices 14 may be any external or implanted medical device. For example, a medical device 14 may be a vital signs monitor or computer, such as a personal digital assistant (PDA) or other handheld computer used to record medical event information while a patient is treated in the field.

As another example, a server 16 may transmit different data destination information 12 for one or more data destinations to a medical device 14 depending on the date or time of day. In some situations, for example, a medical facility may require that defibrillator 30 transmit medical event information 32 via different communication media or networks 22, or to different computers 20 one different days of the week or at different times of the day. This may be due to, for example, network traffic patterns at the medical facility. Similarly, a medical device 14 may select different data destination information 12 stored in a memory 52 for a particular data destination based on the date or time of day. These and other embodiments are within the scope of the following claims.

1. A medical device comprising:
a memory;
a communication interface; and
a processor to store medical event information in the memory during treatment of a patient, receive data destination information from a remote server via a network and the communication interface, and transmit the medical event information to a data destination via the communication interface according to the data destination information.

2. The medical device of claim 1, wherein the processor configures the communication interface according to the data destination information for transmission of the medical event information to the data destination.

3. The medical device of claim 1, wherein the data destination information indicates at least one of a communication medium, a communication protocol, a telephone number, a network address, a network name, or login information for transmission of the medical event information to the data destination.

4. The medical device of claim 1, wherein the data destination information indicates a type of medical event information to be transmitted to the data destination, and the processor transmits the indicated type of medical event information to the data destination.

5. The medical device of claim 1, wherein the data destination information indicates a format for medical event information to be transmitted to the data destination, and the processor transmits the medical event information to the data destination in the indicated format.

6. The medical device of claim 1, further comprising a display, wherein the processor displays at least some of the data destination information via the display.

7. The medical device of claim 1, wherein the processor stores the data destination information in the memory, receives an update to the data destination information from the remote server via the network and the communication interface, and updates stored data destination information based the received update.

8. The medical device of claim 7, wherein the processor provides an indication of a version of the data destination information currently stored in the memory to the server via the communication interface and the network.

9. The medical device of claim 7, wherein the processor receives the update upon powering on of the medical device.

10. The medical device of claim 1,
further comprising a user interface that includes a display,
wherein the memory stores data destination information for each of a plurality of data destinations, and
wherein the processor displays a list of the data destinations via the display, receives a selection of one of the data destinations from the list via the user interface, and transmits medical event information to the selected data destination via the communication interface according to the data destination information for the selected data destination stored within the memory.

11. The medical device of claim 10, wherein the list of data destinations comprises a list of names of facilities that include the data destinations.

12. The medical device of claim 1, wherein the medical device comprises an external defibrillator.

13. A method comprising:
collecting medical event information during treatment of a patient with a medical device;
receiving data destination information at the medical device from a remote server via a network; and
transmitting the medical event information from the medical device to a data destination according to the data destination information.

14. The method of claim 13, wherein transmitting medical event information comprises configuring a communication interface of the medical device according to the data destination information for transmission of the medical event information to the data destination via the communication interface.

15. The method of claim 13, wherein receiving data destination information comprises receiving information that indicates at least one of a communication medium, a communication protocol, a telephone number, a network address, a network name, or login information for transmission of the medical event information to the data destination.

16. The method of claim 13, wherein receiving data destination information comprises receiving information that indicates a type of medical event information to be transmitted to the data destination, and transmitting medical event information comprises transmitting the indicated type of medical event information to the data destination.

17. The method of claim 13, wherein receiving data destination information comprises receiving information that indicates a format for medical event information to be transmitted to the data destination, and transmitting medical event information comprises transmitting the medical event information to the data destination in the indicated format.

18. The method of claim 13, further comprising a displaying at least some of the data destination information.

19. The method of claim 13, further comprising:
storing the data destination information within a memory of the medical device;
receiving an update to the data destination information at the medical device from the remote server via the network; and
updating the stored data destination information based the received update.

20. The method of claim 19, further comprising providing an indication of a version of the data destination information currently stored in the memory to the server via the network.

21. The method of claim 19, wherein receiving an update comprises receiving the update upon powering on of the medical device.

22. The method of claim 13, further comprising:
storing data destination information for each of a plurality of medical facilities within a memory of the medical device;
displaying a list of the data destinations; and
receiving a selection of one of the data destinations from the list,
wherein transmitting medical event information comprises transmitting medical event information from the medical device to the selected data destination according to the data destination information for the selected data destination stored within the memory.

23. The method of claim 22, wherein displaying a list of the data destinations comprises displaying a list of names of facilities that include the data destinations.

24. The method of claim 13, wherein transmitting the medical event information from the medical device to a data destination comprises transmitting the medical event information to a computer of one of a medical facility and a storage facility for the medical device.

25. The method of claim 24, wherein transmitting the medical event information to a computer of a medical facility comprises transmitting the medical event information to a computer of a hospital.

26. A computer-readable medium comprising instructions that cause a programmable processor to:
collect medical event information during treatment of a patient with a medical device;
receive data destination information at the medical device from a remote server via a network; and
transmit medical event information from the medical device to a data destination according to the data destination information.

27. A system comprising:
a plurality of medical devices, each of the medical devices collecting medical event information during treatment of a respective patient; and
a server located remotely from the medical devices that stores data destination information and transmits the data destination information to the medical devices via a network,
wherein the medical devices transmit the medical event information to a data destination according to the data destination information.

28. The system of claim 27, wherein the server transmits different data destination information for the data destination to the medical devices based on a time of day.

29. The system of claim 27,
wherein the server stores data destination information for each of a plurality of data destinations, and transmits the data destination information for each of the plurality of data destinations to each of the medical devices, and
wherein each of the medical devices stores the data destination information for each of the data destinations, displays a list of the data destinations, receives a selection of one of the data destinations from the list, and transmits medical event information to the selected data destination according to the stored data destination information for the selected data destination.

30. The system of claim 27, wherein the server receives a modification to the data destination information from a user, and modifies the stored data destination information based on the received modification.

31. The system of claim 30, wherein the network comprises a first network, and the server receives the modification from the user via a client device and a second network.

32. The system of claim 31, wherein the server presents a web interface including the data destination information currently stored by the server via second network and the client device.

33. The system of claim 31,
wherein the server transmits an update to each of the medical devices based on the received modification, and
wherein each of the medical devices stores the data destination information, and updates the stored data destination information based on the received update.

34. The system of claim 33, wherein each of the medical devices transmits an indication of a version of the data destination information currently stored by the medical device, and the server transmits the update to each of the medical devices based on the indicated versions.

35. The system of claim 27, wherein the server is administered by an emergency medical service.
